(19) United States
(12) Patent Application Publication
(10) Pub. No.: US 2008/0200801 A1
(43) Pub. Date: Aug. 21, 2008

(54) Mapping Movement of a Movable Transducer

(76) Inventors: Douglas Glenn Wildes, Ballston Lake, NY (US); Kenneth Brakeley Welles, Scotia, NY (US); Yury Alexeyevich Plotnikov, Niskayuna, NY (US); Weston Blaine Griffin, Niskayuna, NY (US); Andrzej May, Schenectady, NY (US)

Correspondence Address: GENERAL ELECTRIC CO.;GLOBAL PATENT OPERATION, 187 Danbury Road, Suite 204, Wilton, CT, 06897-4122, US

(21) Appl. No.: 11/677,265

(22) Filed: Feb. 21, 2007

(51) Int. Cl. A61B 5/00; G01D 11/00; G01D 11/30

(52) U.S. Cl. 600/424; 73/866.5

(57) ABSTRACT

A catheter probe includes a transducer configured for at least one cycle of movement within the probe and a sensor that is at least partially supported within the probe for identifying a position of the transducer during the at least one cycle of movement. A method of mapping movement and a test fixture are also presented.

### BACKGROUND OF THE INVENTION

1. Field of the Invention

The subject matter described herein relates generally to calibration and, more particularly, to calibrating movable transducers to avoid distortion.

2. Related Art

Current medical diagnostic demands require reducing an outer diameter of an invasive body such as a catheter to allow the catheter to travel through the narrow and tortuous regions of a patient's vascular system. Contained within the tubular body may be a mechanical rotation system that includes e.g., a motor, a driveshaft, and bearings. The motor may be used to rotate a transducer to acquire a real-time two-dimensional or three-dimensional image. The mechanical rotation system (motor, driveshaft, bearings, ultrasound cables, etc.) may not be “stiff” and may have significant dynamics, causing non-uniform motion and a non-linear relationship between the drive and the actual transducer motion. This, in turn, causes distortion of, and possibly instability in, the ultrasound image, which can make the clinical procedure more difficult, more time-consuming, less safe, or simply impossible. This distortion is sometimes referred to as non-uniform rotational distortion or “NURD”.

To date, no suitable device or method of calibrating an invasive probe to effectively reduce distortion, such as NURD, caused during use of movable transducers is available.

### BRIEF DESCRIPTION OF THE INVENTION

In accordance with an embodiment of the present invention, a method of mapping movement of a transducer within a probe that is configured for sensing within a body cavity, comprises supporting a transducer configured for at least one cycle of movement; providing a sensor; and using the sensor to identify a position of the transducer within the probe during the at least one cycle of movement.

In accordance with another aspect of the present invention, a test fixture for a probe that includes a transducer configured for at least one cycle of movement within the probe comprises a base with at least one probe support extending from the base which, in turn, comprises a probe mount. The test fixture also comprises a sensor that may be interconnected with the at least one probe support for identifying a position of the transducer during the at least one cycle of movement within the probe.

In accordance with a further aspect of the present invention, a catheter probe comprises a transducer configured for at least one cycle of movement within the probe and a sensor that is at least partially supported within the probe for identifying a position of the transducer during the at least one cycle of movement.

### BRIEF DESCRIPTION OF THE DRAWINGS

The following detailed description is made with reference to the accompanying drawings, in which:

FIG. 1 is a diagram, in perspective, showing a test fixture and a probe configured in accordance with an embodiment of the present invention;

FIG. 2 is an enlarged sectional view of a probe configured in accordance with the embodiment of FIG. 1;

FIG. 3 is a diagram showing one particular embodiment of a sensor usable with the probe and test fixture of FIG. 1;

FIG. 4 is a plot of external sensor signal versus external sensor angular position for the test fixture and probe of FIG. 3;

FIG. 5 is a plot of actual and desired angular position of a transducer versus time for the test fixture and probe of FIG. 3;

FIG. 6 is a plot of the difference between the actual and desired angular position of the transducer of FIG. 5, versus time;

FIG. 7 is a perspective view of another embodiment of a sensor usable with the probe of FIG. 1;

FIG. 8 is an end view showing the sensor of FIG. 7 employed with the probe of FIG. 1; and

FIG. 9 is a flow chart showing a method of calibration in accordance with another embodiment of the present invention.

### DETAILED DESCRIPTION OF THE PREFERRED EMBODIMENT

One embodiment of the present invention concerns a device and a method for mapping movement of a transducer within a probe usable to reduce distortions and instability in an ultrasound image and to make the clinical procedure easier, less time-consuming and safer. In the present embodiment, the actual position, e.g. the rotation angle, of a transducer that undergoes at least one cycle of angular movement, is sensed and this information may be used to reduce the distortion, such as non-uniform rotational distortion (NURD). In one particular embodiment, an ultrasound probe is supported by test fixture and a sensor is provided for identifying a position of the transducer throughout the angular cycle of movement.

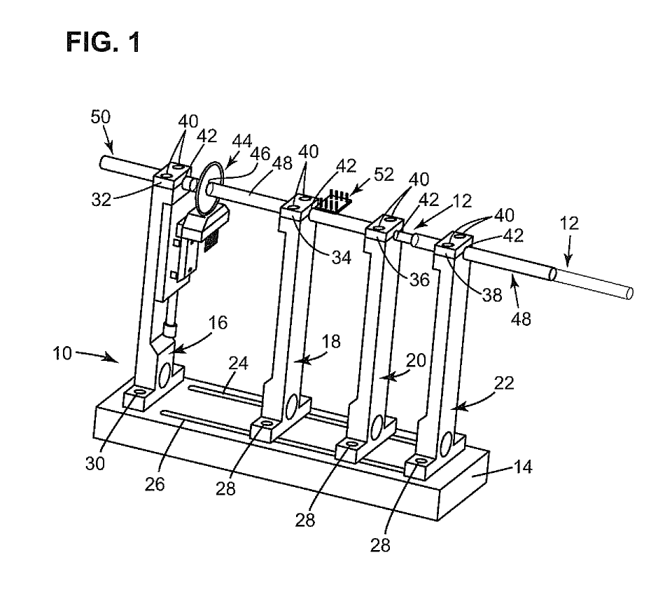

Referring now to FIG. 1, a test fixture and a probe, in accordance with one embodiment of the present invention, are illustrated generally at 10 and 12, respectively. In this embodiment, the test fixture 10 comprises a base 14 and a plurality of probe support members 16, 18, 20 and 22 extending from the base. The base 14 comprises any suitably strong and durable substance such as a metal and, in this embodiment, has a generally stable rectangular outer configuration. The base 14 comprises slots 24 and 26 at which probe support members 18, 20 and 22 are slidably mounted by suitable fasteners 28 for ease of adjustment of space therebetween. Fasteners 30 are provided for mounting the probe support member 16. It will be appreciated that in an optional embodiment, the base 14 may be integral with the probe support members 16, 18, 20 and 22 rather than a separate component and, in another optional embodiment, rather than four probe support members, only two support members (e.g., members 16 and 20) may be provided.

The probe support members 16, 18, 20 and 22 may comprise any suitably strong and durable material similar to that of the base 14 and each comprises a tube mount 32, 34, 36 and 38, respectively. Each tube mount 32, 34, 36 and 38 may comprise a similar material to that of the base 14 and may also comprise fasteners 40 and bushings or bearings 42. It will be appreciated that use of nested support surfaces obviate the use of separate bearings.

In this embodiment, the test fixture 10 also comprises a position indicator such as an optical encoder 44 that includes an encoder wheel 46 that is affixed to a probe support tube 48. The probe support tube 48 may be rotated by an actuator 50. A sensor 52 may be connected to and movable with the probe support tube 48. The sensor 52 may be any suitable sensor that senses an angular position of a transducer located in the probe 12 such as an electromagnetic, magnetic, optical, capacitive, acoustic and/or gravimetric sensor, described in more detail below.

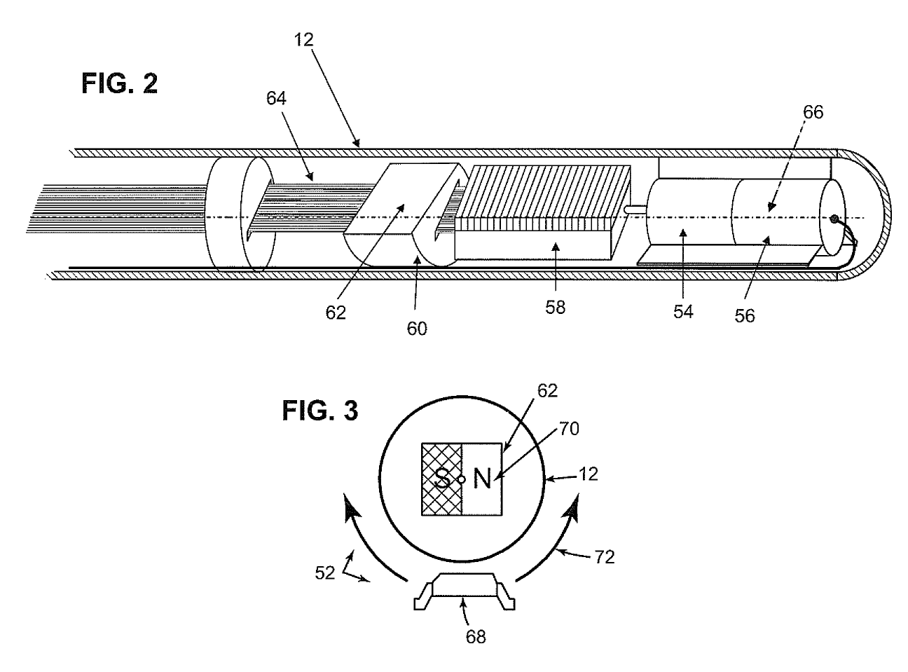

Referring now to FIG. 2, further details of the probe 12 in accordance with one embodiment are shown. In this embodiment, the probe 12 comprises a motor 54, gearbox 56, a transducer 58, a bearing 60, a compartment 62 and a flex cable 64. The motor 54 is known and drives, during operation of the probe 12, a gearbox 56 that, in turn, rotates the transducer 58, about a longitudinal axis 66 of the probe. In one particular embodiment, the transducer comprises an ultrasound transducer array. The compartment 62 rotates along with the transducer 58 and, in one particular embodiment, may be integral therewith. The compartment 62 may contain a magnetic field generator, a reflector, a sensor and/or other device necessary to assist in the sensing and/or to by itself sense (as described in more detail below) an angular position of the transducer 58 throughout a cycle of movement.

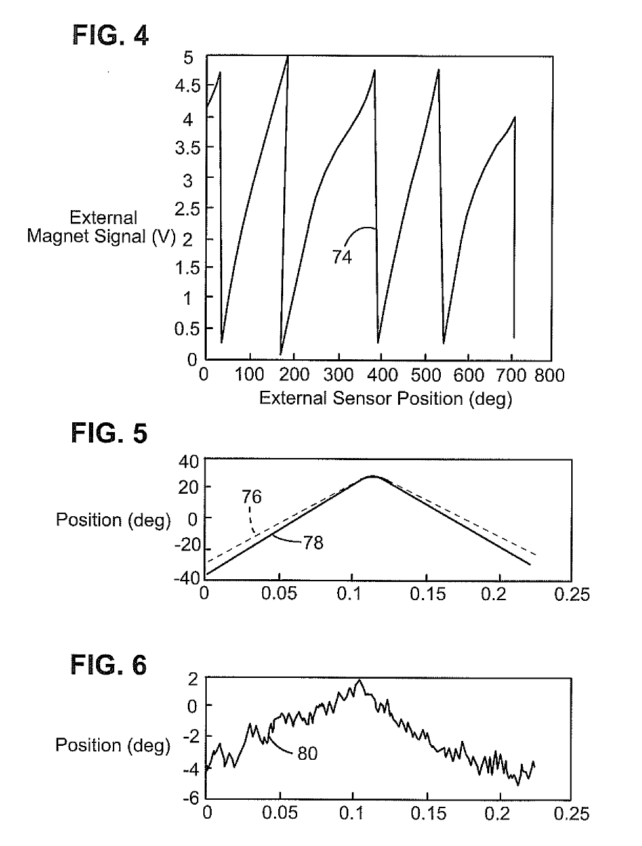

One particular embodiment of the sensor 52 is shown in FIG. 3. In this embodiment, the sensor 52 comprises a magneto resistive sensing element 68 and magnet 70 contained in the compartment 62 of the probe 12. Motor 50 (FIG. 1) rotates the sensing element 68 in either direction shown by arrow 72. In a known manner, this rotation of sensing element 68 about the magnet 70 induces variable output of the sensing element 68 as shown in the plot 74 of FIG. 4 of external sensor signal versus external sensor angular position. The significance of this plot is the sensor output is a smooth monotonic function of the angle between the sensing element 68 and the transducer 58 and magnet 70. The sensor output repeats exactly every 360 degrees. Because the magneto resistive sensor senses the magnitude, but not polarity, of the magnetic field, the sensor output also repeats approximately every 180 degrees (if the magnet is off-center in the tube or the field is in any way asymmetric, the 180 degree repeat will be approximate, not exact). Over the approximately 180 degree range between repeat points, the sensor signal-to-angle relationship may be non-linear, but is highly repeatable, monotonic, and is easily fit and interpolated to give a precise 1:1 transfer function between the sensor output signal and the angle between the magnet (and transducer) and the sensor. With the external sensor in a fixed known position, and the transducer rotating within the probe, applying the transfer function to the sensing element 68 signal output will allow one to determine the angular position of the magnet 70 and thereby transducer 58.

FIG. 5 shows a plot 76 of the angular position of the magnet 70 and thereby the position of the rigidly connected transducer 58 versus time. The plotted angular position 76 of the magnet 70 is determined using the measured sensor signal and the aforementioned transfer function. Plot 78 shows the desired position of the transducer versus time. In FIG. 6, plot 80 shows the difference between plot 76 and plot 78 of FIG. 5, corresponding to the error in the angular position of the transducer versus time. The position error in plot 80, if uncorrected or uncompensated, would cause corresponding image distortion or NURD.

It will be appreciated that the output from the sensor 52 may be included as part of a real-time position control feedback loop. Optionally, if the motion is repeatable, the sensor data could be pre-acquired, processed, and used to modify the drive signals sent to the motor 54 and gear box 56, without real-time feedback.

Referring again to FIG. 2 and in another embodiment of the present invention, the sensor, rather than being disposed outside of the probe 12 as shown in FIG. 1, may be disposed entirely within the probe. In particular, the compartment 62 may contain a sensor that does not require a second component thereof, such as the magneto resistive sensor located outside of the probe described above, to map movement of the transducer. For example, an accelerometer (e.g., a MEMS device) that senses the earth's gravitational field may be mounted within the compartment 62. It is noted that the direction (vector accelerometer) or apparent magnitude (single-axis accelerometer) of the earth's gravitational field varies as the transducer rotates (as long as the axis of rotation is not parallel to the field gradient). In this embodiment, the probe 12 may be rotated along with the accelerometer fixed within the probe to provide mapping of the position of the transducer 58 for calibrating the sensor 52.

Other embodiments of a sensor 52 employable in the practice of the present invention include an electromagnetic coil located in the compartment 62, with the coil axis set nonparallel to the axis of transducer rotation. One or more coils or magnetic field sensors (or a magnet, if there is a coil or sensor on the transducer) are fixed, either internal or external to the probe. Motion of the transducer causes a varying mutual inductance between or induced current or voltage or resistance in the coils or sensors. The relationship between transducer position or motion and the induced electrical signals may be calculated, if the geometry of all components is known and well controlled.

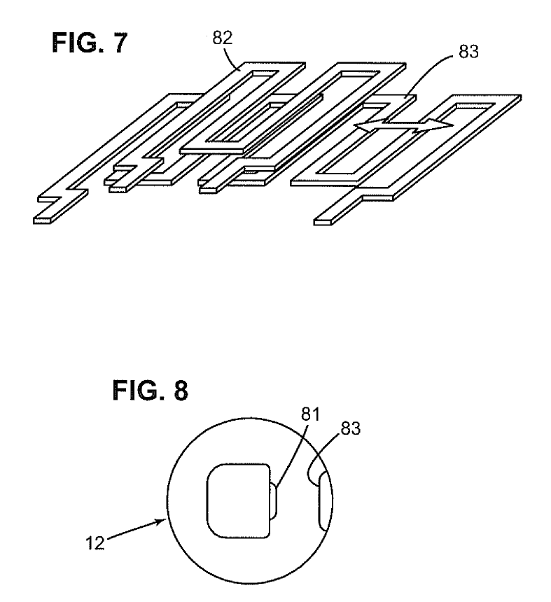

In another embodiment, a magnet and a coil may be employed for sensor 52 which would provide a roughly sinusoidal variation of signal amplitude with rotation angle. To accurately sense the rotation angle, one must measure the relative signal amplitude with high accuracy, which can require calibration, filtering, and long averaging times. Optionally, as shown in FIG. 7, a meander coil 81 may be located in the probe housing or in a calibration device external to the probe housing or on the transducer if it is large enough. An opposing coil 83 is separately mounted to the other of the probe housing, the calibration device or on the transducer. As the transducer moves, the opposing coil 83 moves from one loop of the meander coil 81 or pole of the winding to the next. Each such transition causes a zero-crossing and phase reversal in the output signal, which is much easier to accurately detect than slow variations in the amplitude of a sinusoid. The loops of the meander coil may be of varying dimension. In such a case, the amplitude of the sensed signal would vary with the size of the loop, giving a coarse indication of position (with which loop is the sensor aligned) in addition to the higher-resolution but less clear indication provided by the zero-crossings. The coils can be run at high frequency (e.g, 1 MHz) for increased signal-to-noise and reduced sensitivity to the earth's field, motor drive signals, and other spurious low-frequency signals. As shown in FIG. 8, the meander coil 81 and the opposing coil 83 are located separately on the moving and non-moving components of the probe 12. If the two windings have slightly different spacing or pitch, then a Vernier effect is created with many zero-crossings for much better position resolution.

In a further embodiment of the sensor 52, a capacitive sensor may be employed. For example, a plate of a capacitor may be mounted to the compartment 62 or the transducer 58 itself and a parallel plate may be mounted to a nearby fixed non-rotating portion of the probe 12, such as inside the probe 12. As the transducer 58 is rotated by motor 54, the overlap and thus capacitance of the plates changes, and the position or motion of the transducer can be inferred from an AC measurement of the capacitance. Optionally, the capacitor can be made the frequency-determining element of a resonant circuit and the capacitance and motion derived from measurement of the resonant frequency. It will be appreciated that sensitivity to the motion will be increased and the effects of stray capacitance in the leads reduced, if the spacing between the sensor electrodes is minimized or the electrodes are made of interleaved plates.

In still a further embodiment, the sensor 52 comprises an optical sensor. In one example, a mirror may be located in the compartment 62 adjacent the moving transducer. A laser beam may be directed from a fixed source toward the mirror. A charge coupled device (CCD) or similar high-resolution multi-pixel detector may be positioned to intercept the reflected beam. As the transducer moves, the reflected beam will move across the detector. The relationship between transducer position and detected beam position may be calculated if the geometry is known and well controlled, or may be calibrated, e.g. by holding the transducer fixed within the probe housing and moving the probe relative to the optical system while measuring the probe motion with a linear or rotary encoder.

In another optical sensor embodiment one portion of an optical fiber is attached to the moving transducer, e.g., in the compartment 62, and another portion to a nearby non-moving part of the probe, in such a way that the transducer motion causes the fiber to twist and untwist. An optical signal (laser beam) may be sent through the fiber, either end-to-end or reflected and, it will be appreciated that as the fiber is twisted, the polarization of the transmitted beam will change. From measurements of the change in polarization, one can infer the position or motion of the transducer. Looping the fiber so that multiple strands are twisted in parallel and the optical beam passes through those strands in series could increase the sensitivity of the motion detection.

In still a further optional embodiment, the sensor 52 may comprise an acoustic sensor that may be employed along with a pattern of echogenic targets (high- or low-impedance acoustic reflectors) in the housing of the probe, outside the normal imaging field of view, or in a calibration device external to the probe. For example, if the transducer 58 is rotating within a metal tube catheter tip and imaging through a polymer-covered opening in the tube, the sensor 52 may comprise notches or barbs (projections) provided in the metal at the edges of the opening to create associated dark or bright artifacts in the ultrasound images. In this way, transducer 58 may be used to measure the position of itself. Where the sensor 52 comprises a separate ultrasound array, components external to the probe, e.g., an external housing and/or echogenic target(s) may be provided. Calibration of the sensor 52 may be carried out in a manner similar to that described above, where the external targets could be rotated about a fixed probe to determine a mapping transfer function. With the external target(s) located in a known position, the position of the moving acoustic sensor and thereby the transducer, could be determined using the transfer function applied to the senor output. Additionally, if the geometry of the targets is available, one could incorporate the targets in the non-rotating portion of the probe or, for example, use a non-rotating external target. The targets in the non-rotating portion of the probe may be provided as described above.

In yet a further embodiment of sensor 52, a rotary or linear optical encoder, an LVDT or resolver may be attached to the moving transducer and non-moving portion of the probe 12.

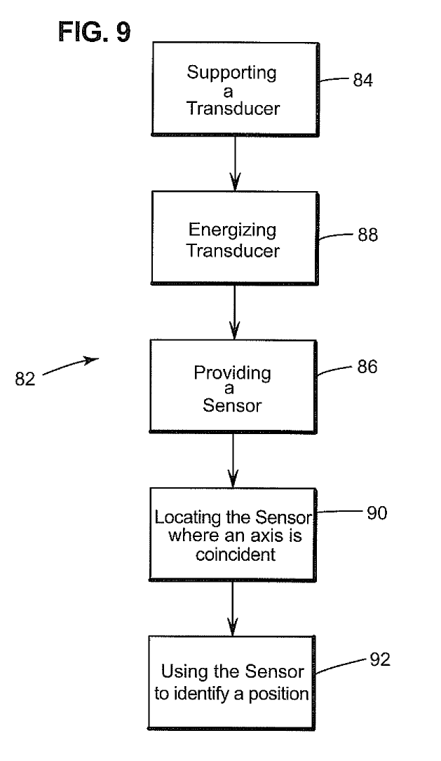

Referring now to FIG. 9, another embodiment of the present invention is shown generally at 82. Specifically, a method of mapping movement of a transducer within a probe, comprises, as shown at 84, supporting a transducer that is configured for at least one cycle of movement. Next, as shown at 86, providing a sensor; and as shown at 92, using the sensor to identify a position of the transducer within the probe during the at least one cycle of movement. The method of mapping movement may also comprise, as shown at 88 energizing the transducer for movement thereof throughout the at least one cycle of movement and sensing the position of the transducer throughout the at least one cycle of movement. It will be understood that the method may be employed where the at least one cycle of movement comprises up to 360 degrees of angular movement or subdivisions thereof, for example, one cycle of movement comprising of a angular movement of approximately 60 degrees.

As shown at 90, the method may further comprise locating a sensor wherein an axis of rotation thereof is coincident with that of the transducer and wherein one step in sensing the position of the transducer comprises moving a sensor about the probe to calibrate the sensor. It will also be appreciated that in one aspect of this method, the sensor may be located within the probe.

While the present invention has been described in connection with what are presently considered to be the most practical and preferred embodiments, it is to be understood that the present invention is not limited to these herein disclosed embodiments. Rather, the present invention is intended to cover all of the various modifications and equivalent arrangements included within the spirit and scope of the appended claims.

What is claimed is:

1. A method of mapping movement of a transducer within a probe that is configured for sensing within a body cavity, comprising:
supporting a transducer configured for at least one cycle of movement;
providing a sensor; and
using the sensor to identify a position of the transducer within the probe during the at least one cycle of movement.

2. The method of claim 1, further comprising energizing the transducer for movement thereof throughout the at least one cycle of movement and sensing the position of the transducer throughout the at least one cycle of movement.

3. The method of claim 1, wherein the at least one cycle of movement comprises up to 360 degrees of angular movement.

4. The method of claim 3, wherein the at least one cycle of movement comprises approximately sixty degrees of angular movement.

5. The method of claim 1, wherein the transducer comprises an ultrasonic transducer.

6. The method of claim 1 further comprising locating a sensor wherein an axis of rotation thereof is coincident with an axis of rotation of the transducer and wherein sensing the position of the transducer comprises moving a sensor about the probe.

7. The method of claim 6, further comprising providing a test fixture for supporting the probe and the sensor and wherein the test fixture comprises an actuator that includes a position encoder and that rotates the sensor.

8. The method of claim 1, wherein the sensor is located within the probe.

9. The method of claim 1, wherein the sensor mode of operation comprises at least one of electromagnetic, magnetic, optical, capacitive, acoustic and gravimetric modalities.

10. The method of claim 9, wherein the sensor comprises a magnet mechanically coupled to the transducer and a magneto resistive element spaced radially from the magnet.

11. The method of claim 4, wherein the sensor comprises at least one echogenic target and wherein the transducer uses reflected signals from the at least one echogenic target to determine its position.

12. A test fixture for a probe including a transducer configured for at least one cycle of movement within the probe, comprising:
a base;
at least one probe support extending from the base, the at least one probe support comprising a probe mount; and
a sensor interconnected with the at least one probe support for identifying a position of the transducer during the at least one cycle of movement within the probe.

13. The test fixture of claim 12, further comprising
a position indicator interconnected with the at least one probe support; and
an actuator interconnected with the position indicator and being configured to rotate the sensor about the probe.

14. The test fixture of claim 12, wherein an axis of rotation of the sensor is coincident with an axis of rotation of the transducer.

15. The test fixture of claim 13, wherein the at least one probe support comprises a plurality of probe supports and wherein each probe support comprises a probe mount, each probe mount comprising a bearing and wherein the test fixture further comprises a probe support tube dimensioned and configured to fit within each probe mount and to receive a probe therewithin, the probe support tube being interposed between the position indicator and the sensor.

16. The test fixture of claim 13, wherein the position indicator comprises an optical encoder wheel that is connected to the probe support tube.

17. The test fixture of claim 16, wherein the sensor mode of operation comprises at least one of electromagnetic, magnetic and optical modalities.

18. The test fixture of claim 16, wherein the sensor comprises a magnet coupled to the transducer and a magneto resistive element spaced radially from the magnet.

19. The test fixture of claim 16, wherein the sensor comprises a mirror coupled to the transducer and an optical sensor element spaced radially from the mirror.

20. The test fixture of claim 12, wherein the sensor identifies the position of the transducer throughout the at least one cycle of movement within the probe.

21. The test fixture of claim 16, wherein the sensor comprises at least one echogenic target and wherein the transducer uses reflected signals from the at least one echogenic target to determine its position.

22. A catheter probe, comprising:
a transducer configured for at least one cycle of movement within the probe; and
a sensor at least partially supported within the probe for identifying a position of the transducer during the at least one cycle of movement.

23. The probe of claim 22, wherein the transducer comprises an ultrasonic transducer and the at least one cycle of movement comprises up to 360 degrees of angular movement.

24. The probe of claim 23, wherein the sensor comprises at least one of an accelerometer, a capacitive sensor, an optical sensor and an acoustical sensor.

25. The probe of claim 23, wherein the sensor comprises a sensor component supported within the probe and wherein the sensor component comprises at least one of a magnet, an electromagnetic coil, and a mirror.

26. The probe of claim 25, wherein the sensor identifies the position of the transducer throughout the at least one cycle of movement.

27. The probe of claim 26, wherein the at least one cycle of movement comprises about sixty degrees of angular movement.
